(19) United States
(12) Patent Application Publication
(10) Pub. No.: US 2025/0060264 A1
(43) Pub. Date: Feb. 20, 2025

(54) Force Detection Device and Force Detection System

(71) Applicants: Tyco Electronics (Dongguan) Ltd., Dongguan (CN); TE Connectivity Solutions GmbH, Schaffhausen (CH); Tyco Electronics (Shanghai) Co., Ltd., Shanghai (CN)

(72) Inventors: Senmiao Hu, Shanghai (CN); Lei (Alex) Zhou, Shanghai (CN); Xuyan Yu, Dongguan (CN); Yi Li, Dongguan (CN); Yong Liu, Dongguan (CN); Dandan (Emily) Zhang, Shanghai (CN); Roberto Francisco-Yi Li, Bellevue, WA (US); Hongzhou (Andy) Shen, Dongguan (CN); Yao Chen, Shanghai (CN)

(73) Assignees: Tyco Electronics (Dongguan) Ltd., Dongguan City (CN); TE Connectivity Solutions GmbH, Schaffhausen (CH); Tyco Electronics (Shanghai) Co., Ltd., Shanghai (CN)

(21) Appl. No.: 18/801,933

(22) Filed: Aug. 13, 2024

(30) Foreign Application Priority Data
Aug. 16, 2023 (CN) 202311037768.1

(51) Int. Cl. G01L 3/14; G01L 5/04; G01N 33/207

(52) U.S. Cl. CPC G01L 3/1478; G01L 5/04; G01N 33/207

(57) ABSTRACT

A force detection device includes a mounting plate, a force sensor installed on the mounting plate, and a lever assembly. The lever assembly is rotatably installed on the mounting plate and is adapted to amplify a pushing force applied thereon and apply the amplified pushing force to the force sensor.

### CROSS-REFERENCE TO RELATED APPLICATION

This application claims the benefit of Chinese Patent Application No. CN202311037768.1 filed on Aug. 16, 2023, in the State Intellectual Property Office of China, the whole disclosure of which is incorporated herein by reference.

### FIELD OF THE INVENTION

Embodiments of the present disclosure relate to a force detection device and a force detection system comprising the force detection device.

### BACKGROUND

In the prior art, it is sometimes necessary to use force to determine whether the quality of a product under test is qualified. For example, force can be used to check whether there is a virtual welding problem in the welded product. However, in high-speed continuous detection, it is difficult for a force sensor to accurately and sensitively detect small forces, and each detection position cannot be clearly identified. This makes it difficult to achieve automated testing. Manual testing, however, has low stability, which affects the automation level of the entire production line.

### SUMMARY

According to an embodiment of the present disclosure, a force detection device includes a mounting plate, a force sensor installed on the mounting plate, and a lever assembly. The lever assembly is rotatably installed on the mounting plate and is adapted to amplify a pushing force applied thereon and apply the amplified pushing force to the force sensor.

### BRIEF DESCRIPTION OF THE DRAWINGS

The invention will now be described by way of example with reference to the accompanying Figures, of which:

FIG. 1 shows an illustrative view of a force detection device according to an exemplary embodiment of the present invention, wherein the mounting plate is in a vertical initial position;

FIG. 2 shows an illustrative view of a force detection device according to an exemplary embodiment of the present invention, wherein the mounting plate is rotated to an inclined position;

FIG. 3 shows an illustrative view of the lever assembly of a force detection device according to an exemplary embodiment of the present invention;

FIG. 4 shows an illustrative view of a lever assembly of a force detection device according to an exemplary embodiment of the present invention, wherein the lever assembly is in its initial position when not in contact with the detected object;

FIG. 5 shows an illustrative view of a lever assembly of a force detection device according to an exemplary embodiment of the present invention, wherein the lever assembly is in a detection position when in contact with the detected object;

FIG. 6 shows an illustrative view of a lever assembly, force sensor, and limiting member of a force detection device according to an exemplary embodiment of the present invention, wherein the limiting member is rotated to a first angle position;

FIG. 7 shows an illustrative view of a lever assembly, force sensor, and limiting member of a force detection device according to an exemplary embodiment of the present invention, wherein the limiting member is rotated to a second angle position;

FIG. 8 shows an illustrative view of a lever assembly, force sensor, and limiting member of a force detection device according to an exemplary embodiment of the present invention, wherein the limiting member is rotated to a third angle position;

FIG. 9 shows an illustrative view of an arm length adjustment device according to an exemplary embodiment of the present invention, wherein the first locking member is in an unlocked position separated from the first screw rod;

FIG. 10 shows an illustrative view of an arm length adjustment device according to an exemplary embodiment of the present invention, wherein the first locking member is in a locking position engaged with the first screw rod;

FIG. 11 shows an illustrative view of a position adjustment device according to an exemplary embodiment of the present invention, wherein the second locking member is in an unlocked position separated from the second screw rod; and

FIG. 12 shows an illustrative view of a position adjustment device according to an exemplary embodiment of the present invention, wherein the second locking member is in a locking position engaged with the second screw rod.

### DETAILED DESCRIPTION OF THE EMBODIMENTS

Exemplary embodiments of the present disclosure will be described hereinafter in detail with reference to the attached drawings, wherein the like reference numerals refer to the like elements. The present disclosure may, however, be embodied in many different forms and should not be construed as being limited to the embodiment set forth herein; rather, these embodiments are provided so that the present disclosure will be thorough and complete, and will fully convey the concept of the disclosure to those skilled in the art.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

According to an embodiment of the present disclosure, a force detection device comprises a mounting plate, a force sensor installed on the mounting plate, and a lever assembly rotatably installed on the mounting plate and can be rotated with its axis of rotation as the fulcrum. The lever assembly is adapted to amplify a pushing force F1 applied to it by a detected object and apply the amplified pushing force F2 to the force sensor.

According to another embodiment of the present disclosure, a force detection system comprises the above force detection device, and a moving device adapted to drive the force detection device to move relative to the detected object.

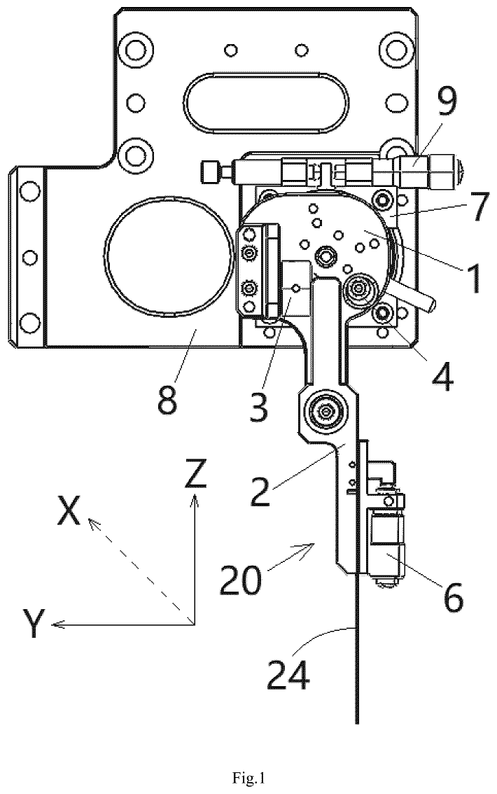

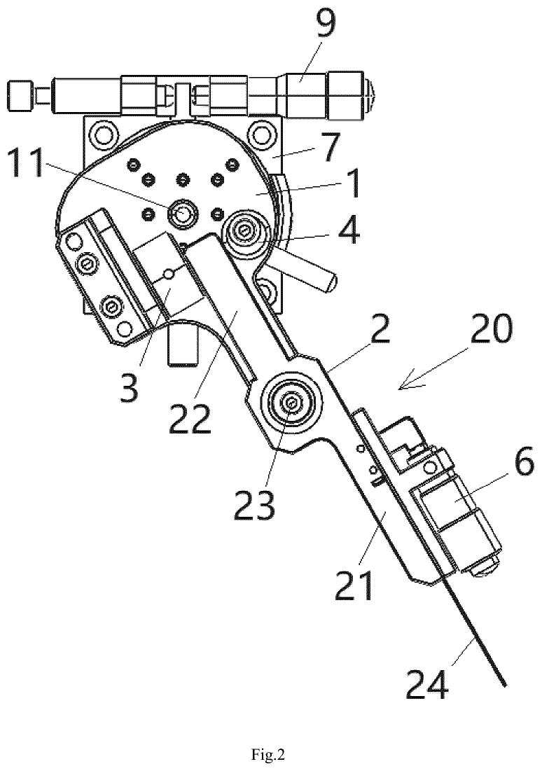

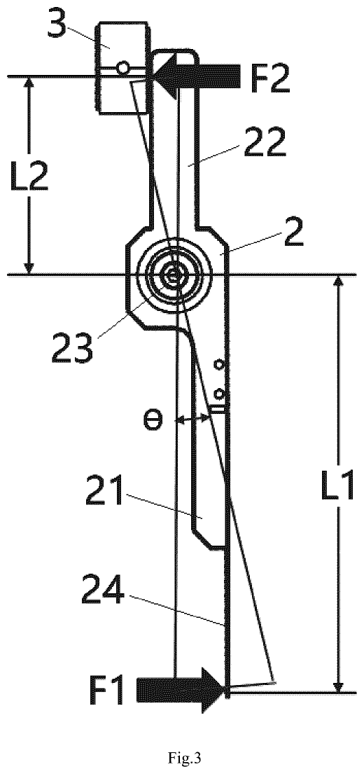

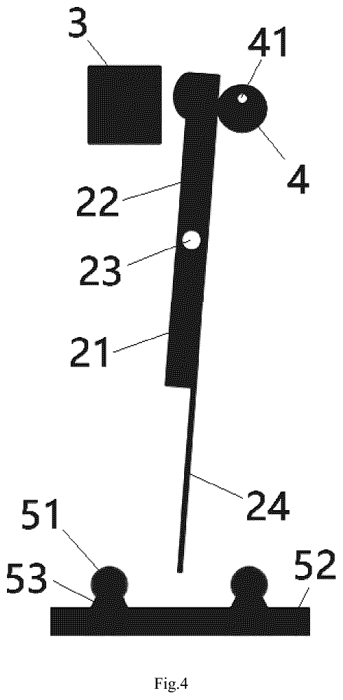

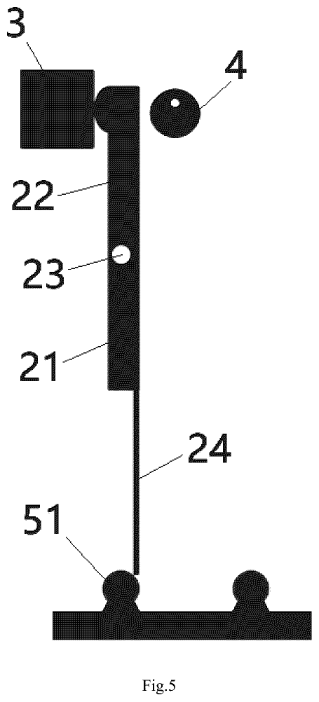

FIG. 1 shows an illustrative view of a force detection device according to an exemplary embodiment of the present invention, wherein the mounting plate 1 is in a vertical initial position. FIG. 2 shows an illustrative view of a force detection device according to an exemplary embodiment of the present invention, wherein the mounting plate 1 is rotated to an inclined position. FIG. 3 shows an illustrative view of the lever assembly 20 of a force detection device according to an exemplary embodiment of the present invention. FIG. 4 shows an illustrative view of a lever assembly 20 of a force detection device according to an exemplary embodiment of the present invention, wherein the lever assembly 20 is in its initial position when not in contact with the detected object 51; FIG. 5 shows an illustrative view of a lever assembly 20 of a force detection device according to an exemplary embodiment of the present invention, wherein the lever assembly 20 is in the detection position when in contact with the detected object 51.

As shown in FIGS. 1-5, in an exemplary embodiment of the present invention, a force detection device is disclosed. The force detection device includes: a mounting plate 1, a force sensor 3, and a lever assembly 20. The force sensor 3 is installed on mounting plate 1. The lever assembly 20 is rotatably installed on the mounting plate 1 and can be rotated with its axis of rotation as the fulcrum 23. The lever assembly 20 is used to amplify the pushing force F1 applied to it by the detected object 51 and apply the amplified pushing force F2 to the force sensor 3.

The lever assembly 20 comprises: a lever member 2 and a movable arm 24. The lever assembly 2 is rotatably installed onto the mounting plate 1. The lever assembly 2 includes a first lever arm 21 and a second lever arm 22 located at both sides of the fulcrum 23, respectively. The movable arm 24 is movably installed on the first force arm 21 and can be moved along the length direction of the first force arm 21. The end of the movable arm 24 extends beyond the end of the first force arm 21 for contact with the detected object 51. The end of the second force arm 22 is used to make contact with the force sensor 3, in order to apply the amplified pushing force F2 to the force sensor 3.

The distance from the end of the movable arm 24 to the fulcrum 23 is referred to as the first arm length L1, and the distance from the end of the second force arm 22 to the fulcrum 23 is referred to as the second arm length L2. The first arm length L1 is greater than the second arm length L2, so that the pushing force F2 applied to the force sensor 3 by the end of the second force arm 22 is greater than the pushing force F1 applied to the end of the movable arm 24 by the detected object 51. In the illustrated embodiments, the ratio of the first arm length L1 to the second arm length L2 is called the force amplification coefficient K, and the force amplification coefficient K can be adjusted by moving the movable arm 24 along the length direction of the first force arm 21.

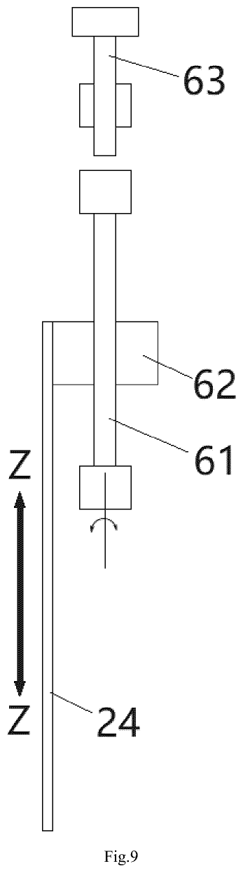

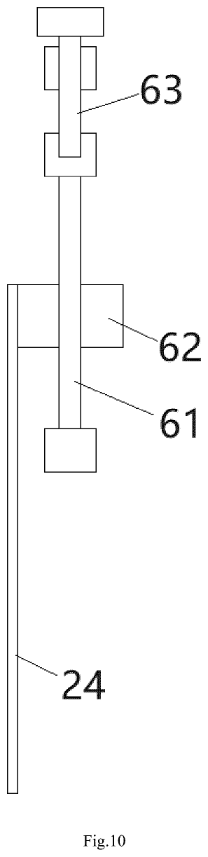

FIG. 9 shows an illustrative view of an arm length adjustment device 6 according to an exemplary embodiment of the present invention, wherein the first locking member 63 is in an unlocked position separated from the first screw rod 61. FIG. 10 shows an illustrative view of an arm length adjustment device 6 according to an exemplary embodiment of the present invention, wherein the first locking member 63 is in a locking position engaged with the first screw rod 61.

As shown in FIGS. 1-5 and 9-10, in the illustrated embodiments, the force detection device further includes an arm length adjustment device 6. The arm length adjustment device 6 is installed on the first force arm 21 of the lever member 2 and is used to move the movable arm 24 along the length direction of the first force arm 21 to adjust the size of the first arm length L1.

The arm length adjustment device 6 comprises a first screw rod 61 and a first nut 62. The first screw rod 61 is rotatably installed on the first force arm 21. The first nut 62 is threaded to the first screw rod 61. The first nut 62 is fixed to the movable arm 24, so that the first nut 62 and the movable arm 24 can be driven to move along the length direction of the first force arm 21 by rotating the first screw rod 61.

The arm length adjustment device 6 further includes a first locking member 63, which is movably installed on the first force arm 21. When the first locking member 63 is moved to the locking position, the first locking member 63 is engaged with the first screw rod 61 to prevent the first screw rod 61 from being rotated; When the first locking member 63 is moved to the unlocked position, the first locking member 63 is separated from the first screw rod 61 to allow the first screw rod 61 to be rotated.

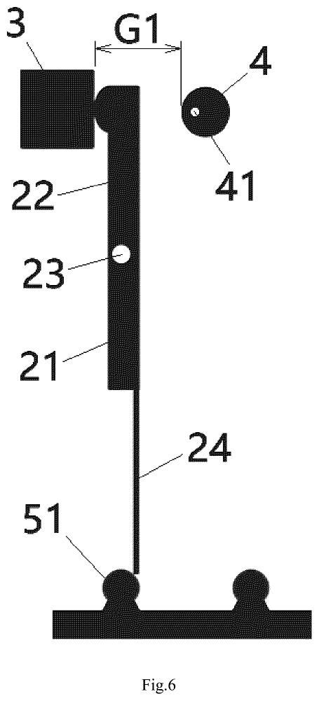

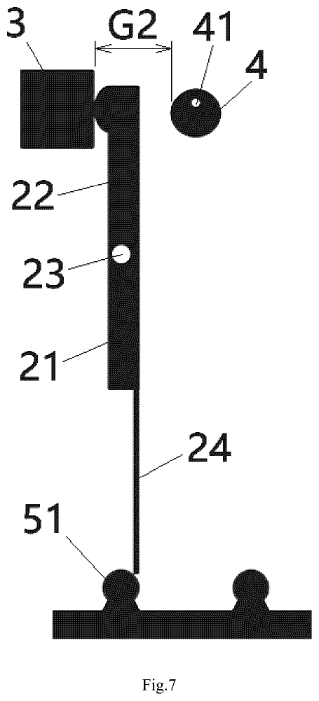

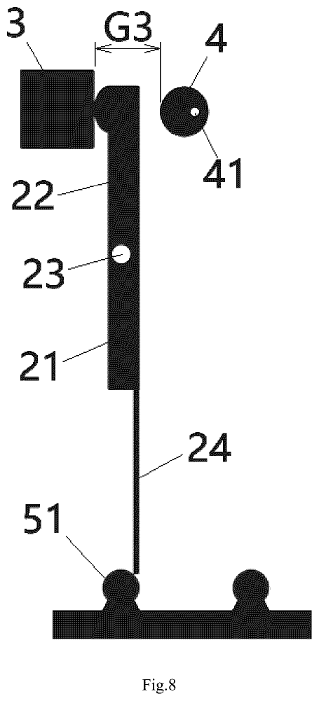

FIG. 6 shows an illustrative view of the lever assembly 20, force sensor 3, and limiting member 4 of a force detection device according to an exemplary embodiment of the present invention, wherein the limiting member 4 is rotated to a first angular position. FIG. 7 shows an illustrative view of the lever assembly 20, force sensor 3, and limiting member 4 of a force detection device according to an exemplary embodiment of the present invention, wherein the limiting member 4 is rotated to a second angular position. FIG. 8 shows an illustrative view of the lever assembly 20, force sensor 3, and limiting member 4 of a force detection device according to an exemplary embodiment of the present invention, wherein the limiting member 4 is rotated to a third angle position.

As shown in FIGS. 1-10, in the illustrated embodiments, the force detection device further comprises: a limiting member 4 and an elastic reset member (not shown, can be a spiral spring). The limiting member 4 is installed on the mounting plate 1 to limit the lever assembly 20 to a predetermined initial position. The elastic reset member is connected between the mounting plate 1 and the lever assembly 20 and is used to reset the lever assembly 20 to its initial position when it is not in contact with the detected object 51.

One end of the lever assembly 20 (the end of the movable arm 24 in the figure) is used to contact the detected object 51, and the other end (the end of the second force arm 22 in the figure) is used to contact the force sensor 3. The other end of lever assembly 20 can be rotated around the fulcrum 23 between the force sensor 3 and the limiting member 4. When the lever assembly 20 is in its initial position, the other end of the lever assembly 20 is against the limiting member 4.

The limiting member 4 can be movably and/or rotatably installed on the mounting plate 1, so that the distance G between the limiting member 4 and the force sensor 3 can be adjusted. The limiting member 4 is an eccentric cam rotatably installed on the mounting plate 1. The eccentric cam can be rotated around its eccentric axis 41 to different angles to adjust the distance G between the limiting member 4 and the force sensor 3.

In the embodiment shown in FIG. 6, the limiting member 4 is rotated to a first angle such that the distance G between the limiting member 4 and the force sensor 3 is equal to a first distance G1. In the embodiment shown in FIG. 7, the limiting member 4 is rotated to a second angle different from the first angle, so that the distance G between the limiting member 4 and the force sensor 3 is equal to a second distance G2. In the embodiment shown in FIG. 8, the limiting member 4 is rotated to a third angle different from the first and second angles, so that the distance G between the limiting member 4 and the force sensor 3 is equal to a third distance G3. In the illustrated embodiment, the first distance G1, the second distance G2, and the third distance G3 are different from each other.

The force detection device further includes a locking member, which can be movably installed on the mounting plate 1. When the locking member is moved to the locking position, the locking member is engaged with the limiting member 4 to lock the limiting member 4, preventing it from being moved or rotated. When the locking member is moved to the unlocked position, it is separated from the limiting member 4 to allow the limiting member 4 to be moved or rotated.

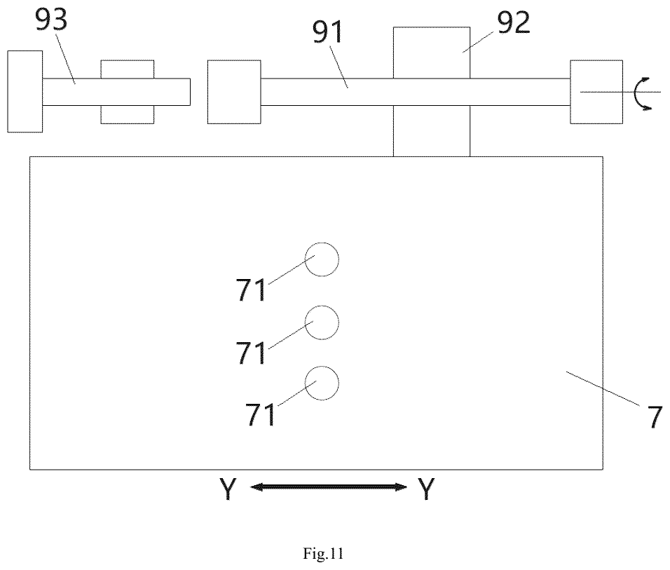

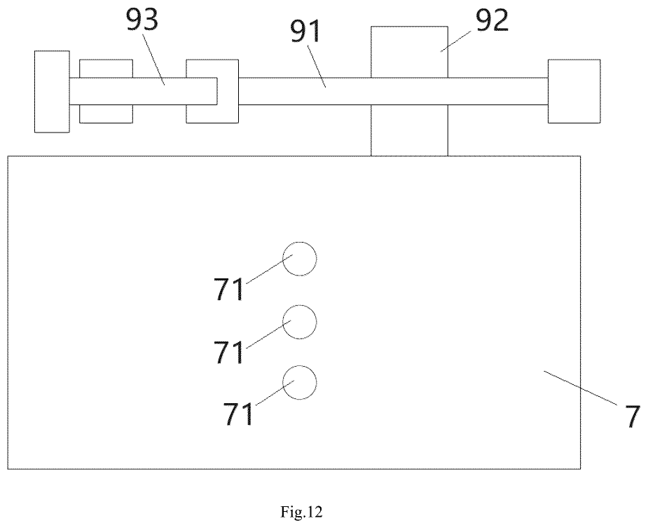

FIG. 11 shows an illustrative view of a position adjustment device 9 according to an exemplary embodiment of the present invention, wherein the second locking member 93 is in an unlocked position separated from the second screw rod 91. FIG. 12 shows an illustrative view of a position adjustment device 9 according to an exemplary embodiment of the present invention, wherein the second locking member 93 is in a locking position engaged with the second screw rod 91.

As shown in FIGS. 1-12, in the illustrated embodiments, the force detection device further comprises a substrate 7. The mounting plate 1 is rotatably installed on the substrate 7 and can be rotated around a horizontal axis parallel to a first horizontal direction X. A pivot hole 71 is formed in the substrate 7, and a pivot shaft 11 is provided on the mounting plate 1. The pivot shaft 11 is rotatably installed in the pivot hole 71, allowing the mounting plate 1 to rotate relative to the substrate 7. Multiple pivot holes 71 are formed in the substrate 7, and the multiple pivot holes 71 are spaced apart in the vertical direction Z. The pivot shaft 11 on the mounting plate 1 can be rotatably installed into any of the multiple pivot holes 71, allowing the position of mounting plate 1 in the vertical direction Z to be adjusted. In the illustrated embodiment, the rotation axis of the lever assembly 20 extends along the first horizontal direction X. The force detection device further comprises a fixed plate 8. The substrate 7 is movably installed on the fixed plate 8 and can move relative to the fixed plate 8 in a second horizontal direction Y perpendicular to the first horizontal direction X.

The force detection device further includes a position adjustment device 9. The position adjustment device 9 is installed on the fixed plate 8 and is used to move the substrate 7 along the second horizontal direction Y to adjust the position of the substrate 7 in the second horizontal direction Y. The position adjustment device 9 comprises a second screw rod 91 and a second nut 92. The second screw rod 91 is rotatably installed on the fixed plate 8. The second nut 92 is threaded to the second screw rod 91. The second nut 92 is fixed to the substrate 7, so that the second nut 92 and substrate 7 can be driven to move in the second horizontal direction Y by rotating the second screw rod 91.

The position adjustment device 9 further includes a second locking member 93. The second locking member 93 is movably installed on the fixed plate 8. When the second locking member 93 is moved to the locking position, the second locking member 93 is engaged with the second screw rod 91 to prevent it from being rotated. When the second locking member 93 is moved to the unlocked position, the second locking member 93 is separated from the second screw rod 91 to allow the second screw rod 91 to rotate.

As shown in FIGS. 1 to 12, in another exemplary embodiment of the present invention, a force detection system is also disclosed. The force detection system includes: the aforementioned force detection device and a moving device (not shown, for example, it can be a translation mechanism). The moving device is used to drive the force detection device to move relative to the detected object 51.

The detected object 51 can be a wire 51 soldered onto the circuit board 52, and the force detection device is used to detect whether the wire 51 has virtual soldering. When the wire 51 undergoes virtual soldering, the solder joint 53 between the wire 51 and the circuit board 52 is not secure and can easily come off. Therefore, at this point, the wire 51 will be detached from the circuit board 52 under the push of lever assembly 20, causing the pushing force change curve detected by the force sensor 3 to be different from the predetermined pushing force change curve. Therefore, it is possible to determine whether the wire 51 has virtual soldering based on the pushing force change curve detected by the force sensor 3. The predetermined pushing force change curve refers to the pushing force change curve detected by the force sensor 3 when the welding quality of wire 51 is qualified.

The distance G between the force sensor 3 and the limiting member 4 of the force detection device is set based on the movement speed V of the force detection device relative to the detected object 51. In the illustrated embodiment, the distance G between the force sensor 3 and the limiting member 4 is inversely proportional to the movement speed V of the force detection device relative to the detected object 51. That is to say, the greater the movement speed V of the force detection device relative to the detected object 51, the smaller the distance G between force sensor 3 and limiting member 4; The smaller the movement speed V of the force detection device relative to the detected object 51, the greater the distance G between force sensor 3 and limiting member 4.

In addition, those areas in which it is believed that those of ordinary skill in the art are familiar, have not been described herein in order not to unnecessarily obscure the invention described. Accordingly, it has to be understood that the invention is not to be limited by the specific illustrative embodiments, but only by the scope of the appended claims.

It should be appreciated for those skilled in this art that the above embodiments are intended to be illustrated, and not restrictive. For example, many modifications may be made to the above embodiments by those skilled in this art, and various features described in different embodiments may be freely combined with each other without conflicting in configuration or principle.

Although several exemplary embodiments have been shown and described, it would be appreciated by those skilled in the art that various changes or modifications may be made in these embodiments without departing from the principles and spirit of the disclosure, the scope of which is defined in the claims and their equivalents.

As used herein, an element recited in the singular and proceeded with the word “a” or “an” should be understood as not excluding plural of the elements or steps, unless such exclusion is explicitly stated. Furthermore, references to “one embodiment” of the present disclosure are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

What is claimed is:

1. A force detection device, comprising:
a mounting plate;
a force sensor installed on the mounting plate; and
a lever assembly rotatably installed on the mounting plate at a pivot and adapted to amplify a pushing force applied thereon by a detected object and apply the amplified pushing force to the force sensor.

2. The force detection device according to claim 1, wherein the lever assembly includes:
a lever member having a first force arm and a second force arm located at both sides of the pivot, respectively; and
a movable arm movably installed on the first force arm and can be moved along the length direction of the first force arm, an end of the movable arm extends beyond an end of the first force arm for contact with the detected object, the end of the second force arm is adapted to make contact with the force sensor to apply the amplified pushing force to the force sensor.

3. The force detection device according to claim 2, wherein a first arm length equal to a distance from the end of the movable arm to the pivot is greater than a second arm length equal to a distance from the end of the second force arm to the pivot such that the pushing force F2 applied to the force sensor by the end of the second force arm is greater than the pushing force applied to the end of the movable arm by the detected object.

4. The force detection device according to claim 3, wherein a force amplification coefficient equal to a ratio of the first arm length to the second arm length is adjustable by moving the movable arm along a length direction of the first force arm.

5. The force detection device according to claim 3, further comprising an arm length adjustment device installed on the first force arm of the lever member, and adapted to move the movable arm along a length direction of the first force arm to adjust the first arm length, the arm length adjustment device including:
a first screw rod rotatably installed on the first force arm; and
a first nut threaded to the first screw rod, the first nut is fixed to the movable arm such that the first nut and the movable arm can be driven to move along the length direction of the first force arm by rotating the first screw rod.

6. The force detection device according to claim 5, wherein the arm length adjustment device further comprises a first locking member movably installed on the first force arm, wherein:
when the first locking member is moved to a locking position, the first locking member is engaged with the first screw rod to prevent the first screw rod from being rotated; and
when the first locking member is moved to an unlocked position, the first locking member is separated from the first screw rod to allow the first screw rod to be rotated.

7. The force detection device according to claim 1, further comprising:
a limiting member installed on the mounting plate to limit the lever assembly to a predetermined initial position; and
an elastic reset member connected between the mounting plate and the lever assembly, and adapted to reset the lever assembly to the initial position when the lever assembly is not in contact with the detected object.

8. The force detection device according to claim 7, wherein:
one end of the lever assembly is adapted to contact the detected object, and the other end is adapted to contact the force sensor;
another end of the lever assembly is rotatable around the pivot between the force sensor and the limiting member; and
when the lever assembly is in the initial position, the other end of the lever assembly is against the limiting member.

9. The force detection device according to claim 8, wherein the limiting member is movably installed on the mounting plate such that a distance between the limiting member and the force sensor is adjustable.

10. The force detection device according to claim 9, wherein the limiting member is an eccentric cam rotatably installed on the mounting plate and rotatable around its eccentric axis to distinct angles to adjust the distance between the limiting member and the force sensor.

11. The force detection device according to claim 7, further comprising a locking member which is movably installed on the mounting plate, wherein:
when the locking member is moved to a locking position, the locking member is engaged with the limiting member to lock the limiting member, so that the limiting member cannot be moved or rotated; and
when the locking member is moved to an unlocked position, the locking member is separated from the limiting member to allow the limiting member to be moved or rotated.

12. The force detection device according to claim 1, further comprising a substrate, the mounting plate is rotatably mounted on the substrate and is rotatable around a horizontal axis parallel to a first horizontal direction, a pivot hole is formed in the substrate, and a pivot shaft is provided on the mounting plate, wherein the pivot shaft is rotatably installed in the pivot hole such that that the mounting plate is rotatable relative to the substrate.

13. The force detection device according to claim 12, wherein:
multiple pivot holes are formed in the substrate and are spaced apart in a vertical direction;
the pivot shaft on the mounting plate can be rotatably installed into any of the multiple pivot holes such that the position of the mounting plate in the vertical direction is adjustable.

14. The force detection device according to claim 12, wherein:
a rotation axis of the lever assembly extends along the first horizontal direction; and
the force detection device further comprising a fixed plate, the substrate is movably mounted on the fixed plate and is movable relative to the fixed plate in a second horizontal direction perpendicular to the first horizontal direction.

15. The force detection device according to claim 14, further comprising a position adjustment device which is installed on the fixed plate for moving the substrate along the second horizontal direction to adjust the position of the substrate in the second horizontal direction.

16. The force detection device according to claim 15, wherein the position adjustment device comprises:
a second screw rod rotatably installed on the fixed plate; and
a second nut threaded to the second screw rod, the second nut is fixed to the substrate such that the second nut and the substrate can be driven to move along the second horizontal direction by rotating the second screw rod.

17. The force detection device according to claim 16, wherein the position adjustment device further comprises a second locking member movably installed on the fixed plate, wherein:
when the second locking member is moved to a locking position, the second locking member is engaged with the second screw rod to prevent the second screw rod from being rotated;
when the second locking member is moved to an unlocked position, the second locking member is separated from the second screw rod to allow the second screw rod to be rotated.

18. A force detection system, comprising:
a force detection device, including:
a mounting plate;
a force sensor installed on the mounting plate; and
a lever assembly rotatably installed on the mounting plate at a pivot and adapted to amplify a pushing force applied thereon by a detected object and apply the amplified pushing force to the force sensor; and
a moving device adapted to drive the force detection device to move relative to the detected object.

19. The force detection system according to claim 18, wherein:
the detected object is a wire soldered onto a circuit board, and the force detection device is adapted to detect whether the wire undergoes virtual soldering;
when the wire undergoes virtual soldering, the wire is detached from the circuit board under the push of the lever assembly, causing the pushing force change curve detected by the force sensor to be different from a predetermined pushing force change curve;
the force detection system determines whether the wire undergoes virtual soldering according to pushing force change curve detected by the force sensor; and
the predetermined pushing force change curve is indicative of a pushing force change curve detected by the force sensor when the welding quality of the wire is qualified.

20. The force detection system according to claim 18, wherein:
a distance between the force sensor and the limiting member of the force detection device is set according to a movement speed V of the force detection device relative to the detected object; and
the distance between the force sensor and the limiting member is inversely proportional to the movement speed V of the force detection device relative to the detected object.
